(19) United States
(12) Patent Application Publication
(10) Pub. No.: US 2024/0285347 A1
(43) Pub. Date: Aug. 29, 2024

(54) ADAPTIVE TARGET REMOVAL FROM SOFT TISSUE

(71) Applicant: Tamar Robotics LTD, Kibbutz Yagur (IL)

(72) Inventors: Tom SHTENDEL, Kiryat-Tivon (IL); Hadas ZISO, Kiryat-Tivon (IL); Noam HASSIDOV, Moshav Bustan HaGalil (IL)

(73) Assignee: Tamar Robotics LTD, Kibbutz Yagur (IL)

(21) Appl. No.: 18/018,626

(22) PCT Filed: Jul. 28, 2021
(86) PCT No.: PCT/IL2021/050912

§ 371 Date: Jan. 30, 2023

Related U.S. Application Data
(60) Provisional application No. 63/057,972, filed on Jul. 29, 2020.

(51) Int. Cl. A61B 34/20

(52) U.S. Cl. CPC A61B 34/20; A61B 2034/2063

(57) ABSTRACT

Systems and methods to correct the working volume of a surgical tool operating under automatic control to ablate within a baseline working volume of tissue subject to shifting and/or shrinkage during ablation. Early ablation may be performed in regions of the target which remain safe with respect to a risk of tissue shifting. At least once after early ablation, the working volume is reset to account for volume shifts, and ablation restarted. In some embodiments, information guiding the working volume reset comprises 2-D imaging results. In some embodiments, early ablation generates fiducial marks used to reset the working volume. In some embodiments, an imager is rotationally coupled to the surgical tool.

### RELATED APPLICATION/S

This application claims the benefit of priority of U.S. Provisional Patent Application No. 63/057,972 filed on 29 Jul. 2020, the contents of which are incorporated herein by reference in their entirety.

### FIELD AND BACKGROUND OF THE INVENTION

The present invention, in some embodiments thereof, relates to the field of surgical devices, and more particularly, but not exclusively, to robotically controlled surgical devices.

A variety of surgeries have been demonstrated (and in some cases, are routinely performed) using robotic systems. Minimally invasive surgical procedures in particular have a potential benefit from appropriately designed robotic manipulators due to considerations, e.g., of size and/or flexibility. Many robotic systems operate under the close guidance of motions by a surgeon-operator.

Neurosurgery is a highly specialized surgical discipline with many further specialized sub-disciplines. Neurosurgical treatment methods have been developed for the removal of tumors and other pathological material, for example, epileptic foci.

International Patent Publication Nos. WO2012/095845 and WO2019/049154 are included herein by reference, in their entirety. These publications relate to robotic systems for neural surgery with the potential for at least partially autonomous operation; e.g., ablation within a predefined surgical volume.

### SUMMARY OF THE INVENTION

According to an aspect of some embodiments of the present disclosure, there is provided a method of guiding a robotically controlled surgical tool to excise a targeted tissue portion within neural tissue, the method including: receiving a first excision plan specifying a first working volume of the targeted tissue portion; excising targeted tissue with the surgical tool with movements limited to within the first working volume according to the first excision plan; and before the targeted tissue portion is fully excised: imaging the targeted tissue portion in its incomplete excised state, generating a second excision plan defining a second working volume, based on the imaging, and continuing excision with the surgical tool according to the second excision plan, and while limited to the second working volume; wherein generating the second excision plan includes: receiving an indication of a shifted boundary of the targeted tissue portion, and defining the second working volume within the targeted tissue portion, based on the indication of the shifted boundary.

According to some embodiments of the present disclosure, the first and second excision plans specify automatic motions of the robotically controlled surgical tool within their respective first and second working volumes.

According to some embodiments of the present disclosure, the automatic motion of the surgical tool is performed according to a progressive cutting pattern.

According to some embodiments of the present disclosure, movements are performed under at least partial manual control, limiting the movements to within at least one of the first and second working volumes includes producing warnings as the surgical tool approaches boundaries of the respective at least one of the first and second working volumes, and responding to the warnings.

According to some embodiments of the present disclosure, the first excision plan is based on pre-operative images.

According to some embodiments of the present disclosure, the indication of a shifted boundary is derived from images obtained by the imaging.

According to some embodiments of the present disclosure, the images the second excision plan is based on indicate boundaries of the targeted tissue portion with adjacent tissue less distinctly than the pre-operative images.

According to some embodiments of the present disclosure, the indication of the shifted boundary includes intraoperatively obtained images of the targeted tissue portion.

According to some embodiments of the present disclosure, the indication of the shifted boundary includes an indication of an excision boundary between excised and non-excised tissue, and the second working volume is defined using the excision boundary as a fiducial mark.

According to some embodiments of the present disclosure, the excision boundary is a boundary between the targeted tissue portion and surrounding neural tissue, and the second working volume is defined along a boundary including the excision boundary.

According to some embodiments of the present disclosure, the second working volume is defined along a boundary offset from the excision boundary.

According to some embodiments of the present disclosure, the offset is determined using the volume of excision already carried out according to the first excision plan.

According to some embodiments of the present disclosure, movement of the excision boundary is tracked while excising according to the first excision plan, and the second working volume is defined also using tracked movement of the excision boundary.

According to some embodiments of the present disclosure, excising according to the first excision plan is stopped until the second excision plan is generated.

According to some embodiments of the present disclosure, the second working volume is defined by one or more 2-D perimeters corresponding to 2-D cross-sections of the targeted tissue portion.

According to some embodiments of the present disclosure, the one or more 2-D perimeters are defined by a plurality of anchor points.

According to some embodiments of the present disclosure, the one or more 2-D perimeters are defined by manual selection performed in conjunction with visual display of images obtained by the imaging.

According to some embodiments of the present disclosure, the one or more 2-D perimeters are defined by automatic segmentation performed on images obtained during the imaging, wherein the automatic segmentation is further based on: the movement of image features identified in a plurality of 2-D images obtained during the excising, and reference 2-D perimeters corresponding to the estimated position of the targeted tissue portion pre-excision.

According to some embodiments of the present disclosure, the one or more 2-D perimeters are defined by automatic segmentation performed on images obtained during the imaging, wherein the automatic segmentation is further based on: comparison of an excised region shown in the images with a region corresponding to a cross-section, within a plane of the image, of robotic positions during movements carried out during the excising.

According to some embodiments of the present disclosure, the imaging is performed using an ultrasound imager.

According to some embodiments of the present disclosure, the excising proceeds through a series of layers, stacked along a longitudinal axis of the surgical tool.

According to some embodiments of the present disclosure, the excising proceeds through a series of sections oriented substantially parallel to a longitudinal axis of the surgical tool.

According to some embodiments of the present disclosure, the sections are slab- or wedge-shaped regions.

According to an aspect of some embodiments of the present disclosure, there is provided a system for automatic excision of a tissue portion within neural tissue, including: a robotically operated surgical tool; a robotic controller configured to: receive an excision plan including a defined working volume, and automatically operate the surgical tool to excise tissue within the working volume; an imager, operable to obtain images of the tissue portion during operation of the surgical tool to excise tissue; and a target tissue tracker including a processor programmed to: define, using images obtained with the imager, a changed position of the tissue portion after movement due to operation of the robotically operated surgical tool, generate, based on the changed position, a new excision plan including a new working volume, and provide the robotic controller with the new excision plan to continue automatic operation of the surgical tool to excise tissue within the new working volume.

According to some embodiments of the present disclosure, the target tissue tracker also defines the changed position of the tissue portion using user-generated selections guided by the images obtained with the imager.

According to some embodiments of the present disclosure, the robotically operated surgical tool includes a longitudinally extended device having a flexible and steerable tip with a cutting end.

According to some embodiments of the present disclosure, the imager is rotatingly coupled to the robotically operated surgical tool to allow rotation of the imager around a longitudinal axis of the surgical tool.

According to some embodiments of the present disclosure, the imager is attached to an angled mount, and sized for insertion to a sub-cranial space; the image is rotatingly coupled to the surgical tool through an attachment of the angled mount.

According to some embodiments of the present disclosure, the imager is attached to an outer surface of the cannula.

According to some embodiments of the present disclosure, the imager is held within an inner lumen of the cannula.

According to some embodiments of the present disclosure, the cannula includes a laterally positioned window, sized to allow a distal tip of the surgical tool protrude through.

According to some embodiments of the present disclosure, the imager is an ultrasound imager.

According to some embodiments of the present disclosure, the imager images the tissue portion through a selectable planar section thereof, and the robotic controller is configured by the excision plan to move the surgical tool through a pattern that excises tissue throughout a first selected planar section imaged by the image, before moving to excise tissue in an at least second selected planar section.

According to some embodiments of the present disclosure, the first selected planar section extends substantially parallel to a longitudinal axis of the surgical tool.

According to some embodiments of the present disclosure, within each selected planar section, the robotic controller is configured to move the surgical tool distally and proximally along a longitudinal axis of the tissue portion, and radially outward, while successively excising a plurality of layers of tissue.

According to an aspect of some embodiments of the present disclosure, there is provided a system for automatic excision of a tissue portion within neural tissue, including: a robotically operated surgical tool; a robotic controller configured to: receive an excision plan including a defined working volume, and automatically operate the surgical tool to excise tissue within the working volume; an imager, operable to obtain images of the tissue portion during operation of the surgical tool to excise tissue; and a processor programmed to present an indication of risk to non-targeted tissue, based on the images and the excision plan.

According to some embodiments of the present disclosure, the indication of risk includes at least one image of tissue within the working volume, presented along with an indication of the relative position of boundaries of the defined working volume.

According to some embodiments of the present disclosure, the image is a 2-D image, boundaries of the defined working volume are presented a 2-D cross-sectional form.

According to some embodiments of the present disclosure, the image is an ultrasound image.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIGS. 1A-1D schematically represent a model of internal forces within a soft tissue (e.g., brain tissue), according to some embodiments of the present disclosure;

FIGS. 2A-2J represent movements of tissue portion under compression in response to hollowing-out, according to some embodiments of the present disclosure;

FIGS. 3A-3J schematically represent a method of image-guided adjustment of a working volume for a surgical tool, according to some embodiments of the present disclosure;

FIG. 3K is a flowchart of a method of image-guided adjustment of working volume optionally corresponding to what is illustrated in FIGS. 3A-3J, according to some embodiments of the present disclosure;

FIGS. 3L-3M schematically represent adjustment of a working volume comprising a blood vessel for a robotic surgical tool, according to some embodiments of the present disclosure;

FIGS. 4A-4C schematically represent an imaging pattern which may be used to monitor tissue cutting, according to some embodiments of the present disclosure;

FIG. 5A schematically illustrates a large-access configuration for imaging a target tissue portion using an imager head, according to some embodiments of the present disclosure;

FIGS. 5B-5C schematically illustrate a reduced-size access configuration for imaging a target tissue portion using an imager head, according to some embodiments of the present disclosure;

FIG. 5D schematically illustrate a cannula-mounted imager head, according to some embodiments of the present disclosure;

FIGS. 6A-6B schematically represents a cannula-mounted imager head, according to some embodiments of the present disclosure;

FIG. 7 schematically illustrates a system for target-adjustable treatment of tissue by a robotically operated surgical tool, according to some embodiments of the present disclosure;

FIG. 8 is a flowchart of a method of risk-guided excision of tissue, according to some embodiments of the present disclosure; and

FIG. 9 schematically represents a time series of movement pattern of a cannula and surgical tool to excise a targeted tissue portion from within a larger tissue region, according to some embodiments of the present disclosure.

### DESCRIPTION OF SPECIFIC EMBODIMENTS OF THE INVENTION

The present invention, in some embodiments thereof, relates to the field of surgical devices, and more particularly, but not exclusively, to robotically controlled surgical devices.

### Overview

An aspect of some embodiments of the present disclosure relates to the guidance of a robotically controlled surgical implement according to parameters that define a safe envelope of movement, even though the envelope itself is dynamic. In some embodiments, the protection comprises restriction of movements. In some embodiments, the protection comprises indications of approaches to and/or violations of a selected envelope of movement.

Within the context of neurosurgical applications, the safe envelope for movements can be extremely narrow, compared to the size of a target of neurosurgical intervention. Within some regions, even a small area of accidental damage can produce serious loss of neural function, up to and including death. In neurosurgical resection and/or fluid removal procedures—for example, in a procedure for removing a tumor, a hematoma, or an epileptic focus—the safe envelope of movement can be practically synonymous (e.g., bounded within 1 mm of) with the extent of the excision target itself. Potentially, at least some of the excision target's boundaries are explicitly defined according to a maximum safe extent of excision.

Moreover, surgical activity itself can change the safe envelope of movement. Brain tissue may move, for example, in response to changes in intracranial pressure; even surgically accessing the cranial cavity can induce shift. Removal of tissue may effectively create localized pressure and other changes in force distribution that disturb tissue shape, regardless of attempts to maintain a constant pressure environment (e.g., with saline and/or another fluid replacement for removed tissue). Surrounding tissue may move into gaps created by tissue excision. Alternatively, tissue may move away from gaps, for example if fluid in the gaps becomes relatively pressurized, or if excision releases local elastic tension. The gap itself may be displaced if tissue on one side expands more than on the other. For example, a thin tissue layer on one side of an excision may not expand as much as a thicker tissue layer on the other. The movements can be conceptualized by a spring and dashpot model, wherein changes to the target generate an imbalance of forces which exert to gradually reach a new equilibrium position. This model, however, should not be understood to not exclude the potential for “jumps”, for example, at moments when forces are suddenly released.

In view of these considerations, a potential problem for the robotic automation of neurosurgical procedures (and particularly neurosurgical resection procedures) is that safe envelope of movement is both subject to stringent tolerance requirements, and dynamic. Human supervision can in principle assist, but even in this case, a problem remains of how to balance human judgement with robotic precision to efficiently and reliably produce a safe surgical outcome. Selection of an appropriate such balance can depend on properties of the robotic surgical instrument being used.

In particular, robotic surgical instruments operated in a “keyhole” configuration may provide benefits such as helping to minimize incidental surgical trauma. Keyhole procedures may be used to access deep brain structures via a cannula with limited or no option for sideways movements of the cannula aperture after cannula insertion.

In some embodiments of the present disclosure, such an instrument operates incrementally to treat a target (e.g., a target embedded in brain tissue), with the result that a procedure comprises a large number of fine movements executed within a damage-sensitive environment. To the degree it is practicable, automation of these movements potentially reduces operator training needs, operator fatigue, errors, and/or the duration of the procedure itself.

Recalling, however, that the safe envelope and/or target of instrument movement may be dynamic, this incremental approach leads to a problem of how to maintain robotic operations within the safe envelope of movement. In some embodiments of the present disclosure, solutions to this problem comprise defining a working volume designated for excision movements; and periodically redefining the working volume, based on an assessment that a level of risk has been reached comprising risk of excising non-targeted tissue. More particularly, in some embodiments of the present disclosure, there are provided one or more methods of intraoperatively reconfiguring automatic movements and/or safe zone limits on automatic movements of a robotic device according to movements (tissue shifts) of an excision target; for example movements as may be induced by excision during an earlier phase of the operation.

Herein, the term “working volume” refers to a volume within which a tissue-dissecting portion of a robotic surgical tool is presently allowed to execute tissue-dissecting movements. The tissue-dissecting portion of the surgical tool, in some embodiments, comprises working surfaces of the surgical tool which contact tissue to detach it from surrounding tissue; e.g., by slicing, cutting, and/or abrading. Herein, the terms “excise” “excision” and “excising” are also used to generically describe tissue dissection, though this convention does not exclude that the term “cut” may be used without excluding another mode of dissection such as slicing or abrading.

In some embodiments, the tissue-dissecting portion of the surgical tool comprises a surface to which energy (e.g., heat and/or electromagnetic energy) is applied to controllably dissect adjacent tissue. In some embodiments, the tissue-dissecting portion of the surgical tool comprises an aperture through which energy is applied (e.g., pressure and/or a jetting fluid) to controllably dissect adjacent tissue. In some embodiments, the working volume is defined in conjunction with a movement pattern of the robotic surgical tool so that some portions of the working volume are predictably reached earlier and some portions predictably reached later. All these portions are “presently allowed”, in the sense that the pattern, if allowed to complete, will sweep through the whole of the working volume including those portions. However what is “presently allowed” may result in the destruction of untargeted tissue, if tissue within and/or surrounding the presently allowed working volume has moved since it was earlier defined.

As further way of understanding the scope of “presently allowed”: the working volume may be defined by a boundary between “go” and “no go” zones, the working volume being the “go” zone. Movements, including excision of tissue, within the go zone are considered safe and/or permitted. Movements that would exceed the bounds of the go zone are considered unsafe and/or non-permitted. Bounds-exceeding movements are either halted or warned of (e.g., in the case of manual control of the robotic surgical tool); or simply avoided (e.g., by automatically controlled movements of the robotic surgical tool).

It is noted that the surgical tool may have other portions which mount and/or manipulate the tissue-dissecting portion. Those tool portions closest to the tissue-dissecting portion will generally be constrained to stay within pre-existing open areas and/or previously-excised volumes, at least once excision within the region of targeted tissue has begun.

In some embodiments, the working volume provides an input to an automatic robotic controller, which the controller uses to further define the bounds within which particular robotic motions will be defined and carried out (e.g., according to a general pattern adapted for the specifics of the target size and shape). In some embodiments, manual control of a robotic surgical tool is guided to remain within the working volume by the use of indications and/or warning; for example, based on instantaneous position, movement vectors, and/or user-specified point-to-point robotic motions.

An aspect of some embodiments of the present disclosure relates to the use of images to assist assessment of the risk that non-targeted tissue is imperiled by excision, due to movement of the non-targeted tissue into a current working volume. In some embodiments, inputs to risk assessment are gathered using one or more of a computerized user interface and an imaging device.

As excision proceeds, initial conditions potentially change (e.g., due to tissue shift), so that the initial assumption that the working volume is safe becomes increasingly uncertain. In particular, the working volume may begin to have non-target tissue in it. At some point, the previously accepted safety of excising within the working volume or some portion of it may be recognized as invalid (that is, there becomes too much risk to non-target tissue to proceed). This recognition may be arrived at automatically and/or by manual intervention, e.g., based on analysis and/or review of images of the progress of excision. The recognition optionally is followed by a halt and/or restriction in excision operations to allow redefine the working volume according to new conditions; after which tissue excision resumes as normal.

In some embodiments, risk assessment is manual, based on inspection of intra-procedural images. In the case of neurosurgical procedures, soft tissue contrast may be so limited for many imaging modalities (e.g., ultrasound), that it potentially falls below the reliable limits of identification by machine vision algorithms. Higher resolution and/or higher contrast imaging devices are potentially expensive, difficult to use routinely in the course of surgery, and/or based on the use of potentially harmful radiations (radioactive tracers and/or high frequency electromagnetic radiation).

However, use of computerized algorithms in not only presenting indications of risk, but optionally also assessing risk based on such indications, may nevertheless be of use, at least in some circumstances. In some embodiments, indication of risk assessment uses computerized algorithms to detect (and optionally at least partially predict) target movements; and/or to adjust a working volume of the surgical instrument. Outputs of the computerized algorithms are optionally confirmed and/or modified by a human operator.

An aspect of some embodiments of the present disclosure relates to assessment of risk and/or planning of excisions based on risk dynamics due to tissue shifts during a tissue excision procedure.

In some embodiments, successive working volumes are defined in response to changes in risks; e.g., working volumes are larger for likely low-shift portions of a procedure, and smaller during portions of a procedure when larger shifts are more likely. In some embodiments, a working volume is offset from center within a larger volume of tissue targeted for removal, partially compensating for an anticipated later shift.

An insight of the inventor is that in excision treatments associated with potentially unpredictable intraoperative tissue dynamics, both uncertainty and the margin for error may be themselves dynamic. This allows optional tradeoffs and/or optimizations to be made in the degree of autonomy that automatically controlled excision can be granted.

First, target movement develops over a period of time, and not even necessarily at a constant rate. Pressure imbalance caused by tissue removal, for example, may induce at first very little motion (e.g., insofar as remaining structural integrity resists the imbalance), then induce a maximum motion (e.g., a collapse or shift), and finally, as equilibration is approached, taper off to small or no motions. Autonomous excision may be relatively safe (e.g., easily stopped in time) during periods of slower movement. Even rapid movement may be safe if the effect is to move tissue into the path of surgical excision which was anyway targeted for removal—that is, if there remains sufficient “buffer” tissue, at least in the direction from which tissue shift is most likely.

Second, target movement is not unlimited in magnitude—to a good approximation, it is limited by the volume of tissue already removed, and may be instantaneously even smaller, since movements may lag their induction by tissue removal. This in turn affects safety considerations for planning robotic movements—as “buffer” size decreases, there may also be a corresponding decrease in the likely size and/or rate of further uncontrolled tissue movements.

Other aspects of tissue movement that may be partially predictable include, for example, expecting less expansion from a side of an excised volume nearer to a solid boundary such as cranial bone, compared a side beyond which there exists a substantial volume of fluid and/or soft tissue (e.g. cerebrospinal fluid and/or brain). This biases the likely direction of tissue movement toward the direction of the solid boundary. Working volumes and/or movement patterns within those working volumes may be selected to take this likely direction into account, to maintain relative larger buffers of safe-to-excise tissue on the side from which movement is most likely, as long as possible.

There may also be more or less critical boundaries of the safe envelope of movement of the surgical tool. For example, a speech-critical area (e.g. Broca's area) may lie on one side of tissue targeted for excision, with primary motor cortex on the other side. Even while preferable to avoid damage to either, the decision may be made that damage to the imperiled region of primary motor cortex is a less disfavored outcome. Errors in relative positioning of surgical tool and target tissue, should they occur, are optionally biased (e.g., by the selection of a pattern of tool movements) to occur nearer to less critical boundaries.

An aspect of some embodiments of the present disclosure relates to the decoupling of working volume definition from iterative motions of an automatically and robotically controlled excision procedure.

In some embodiments, all or most motions of the robotic surgical tool occur without direct feedback control; instead postponing control to be based on periodic reassessments of working conditions (such as tissue shifts) within and/or around the current working volume. In some embodiments, accordingly, excision activity (tissue dissection) is constrained to a working volume (also referred to herein as a “go zone”) which is, at least initially, expected to remain within the volume of targeted tissue. If there are tissue shifts that eventually invalidate this expectation, excision activity may be halted and/or restricted (e.g., restricted to avoid advancing within the working volume); and optionally restarted and/or released from restrictions after redefining the working volume.

In some embodiments, the working volume is defined to be just the same as the initial volume of targeted tissue, and work halted when observed tissue shifts (e.g., observed by use of imaging concurrent with tissue excision) create mismatch that imperils non-targeted tissue. Alternatively, the working volume is initially created to be undersized compared to the targeted tissue volume.

In some embodiments, a predictable pattern of movement of a robotically controlled surgical tool is used for excision, the pattern being selected so that even after initial tissue shift, excision work may optionally continue until mismatch becomes so great that non-targeted tissue is imperiled (e.g., as automatically determined and/or in the judgement of a device operator), and/or until the moving location of tissue excision begins to imperil non-targeted tissue. Optionally, the working volume (in view of a certain planned pattern of excision movements) is initially defined to anticipate at least some portion of tissue shift (e.g., its approximate direction and/or magnitude), potentially helping excision work remain within the targeted volume for a longer period before adjustment is needed.

In some embodiments, a procedure is divided into at least an earlier phase and a later phase. In the earlier phase, a working volume for excision activity confines itself, e.g., to a central portion of the target volume, defined such that tissue shift which may plausibly occur will at worst bring a boundary of the target volume to an edge of the central portion. During this phase, the robot can work automatically and optionally even “blindly”; since as long as it remains within the “go zone”, it doesn't affect the final result if it excises tissue that was anyway targeted for eventual removal, even though not originally within its working domain.

Alternatively, in some embodiments, the working volume set during the early phase includes areas that may become unsafe (e.g., due to tissue shift) before the working volume is fully visited. For example, the working volume may include the whole of the targeted tissue volume, regardless of the potential for tissue shift. However, the pattern of automatically planned movements to excise tissue within the working is itself defined so that there is unlikely to be a combination of sudden surgical tool movements and tissue shifts which imperil non-targeted tissue without sufficient warning to halt excision work (e.g., at least enough time to take a next monitoring image and/or a time longer than the judgement and reaction time of an operator). In such embodiments, the ordinary early-phase procedure may be to allow excision to continue until surgical tool position and the boundary of the targeted tissue volume are observed, judged, and/or anticipated to reach a potentially unsafe proximity.

In the later phase, the working volume is optionally adjusted to define a new “go zone”, after which excision continues. Optionally, the robot can again work blindly for a period (e.g., a period comprising excision of at least one layer of a progressive pattern, for example a longitudinally, laterally, and/or radially progressive pattern, as described hereinbelow). The process can repeat iteratively, if necessary, until reaching the outer margins of the target volume. Later excision activity has a potentially lower margin for error (since it may be happening closer to the edges of the safe envelope of movement). But since the remaining capacity for movement remodeling is also reduced, the level of uncertainty is also potentially reduced. Optionally one or more phases of manual control of robotic motions may be performed to complete excision to the outer margins of the target volume. Work during manual control of robotic motions is optionally limited to within the working volume by the production of warnings when a motion causes the surgical tool to approach a boundary of the working volume, to which the manual operator is trained to respond by halting and/or redirecting tool movements.

This method may be suited in particular to use with low-contrast intraoperative imaging, which may be otherwise insufficient as a data source to guide fully dynamic (e.g., real-time evaluated) and/or automatic updating of a working volume in response to tissue movements.

An aspect of some embodiments of the present disclosure relates to automatic detection of increased risk of excision of non-targeted tissue, using boundaries of previously excised tissue regions.

In some embodiments, boundaries between excised and un-excised tissue themselves become a source of image contrast; for example, excised tissue may be replaced (e.g., by irrigation, optionally after aspiration) with a fluid material (e.g., saline, or another liquid or gas) which has a higher contrast with soft tissue than the original soft tissue target has with surrounding tissue. In the latter case, a “blind” first phase is optionally followed by a period of more closely image-guided excision activity, relying on inferences anchored by clearer images of the excised volume. A potential drawback to this approach is that fragments of excised material may remain in the excavated cavity. When such fragments migrate to the edges of the currently excised volume, they potentially obscure the boundary, since they can have similar contrast to the surrounding material.

In some embodiments, consequences of this effect are addressed by obtaining a plurality of images during careful flushing and/or stirring of the replacement fluid to induce movement of fragments in the excised region. Image areas which show changes from image to image (a motion signal) may be considered as imaging the region where excision has already taken place. In some embodiments, small particles (e.g., latex beads) may be introduced to deliberately induce inter-image changes. In either case, the changes induced between images may be understood as comprising a motion contrast signal which potentially distinguishes a volume in which fluid can mix freely from surrounding, more solid regions. Motion signal analysis may be combined with raw contrast measurements, and optionally other constraints (e.g., knowledge of the edge shapes of the excised region).

An aspect of some embodiments of the present disclosure relates to the design of movement plans of a robotic surgical tool to help manage risk of inadvertent excision of non-targeted tissue.

In some embodiments, the robotic surgical tool executes automatically controlled movements within the working volume to dissect tissue targeted for excision according to a movement plan (also called herein an “excision plan”, for surgical excision procedures). The term more particularly relates to a specifying feature of an excision plan which defines movement of a surgical tool throughout the working volume so that tissue in substantially all parts of the working volume will be excised (typically, mechanically destroyed, and preferably removed from the working volume).

In some embodiments, an excision plan defines a progressive pattern of movements that advances the excising zone gradually toward the go/no go boundary. Progressive movement has potential advantages for predictability, which in turn may allow working volume specification to be active for a longer period before the margin of safety is reduced to the point where a redefinition is needed.

One example of such a pattern (a “longitudinally progressive” pattern) sweeps a tissue-dissecting portion of a surgical tool through a sequence of tissue layers, covering a whole substantially flat surface in each layer before advancing to excise the next layer. In some embodiments, a bore-hole is excised just to about the middle of the working volume, and then excision continue radially outward (“radially progressive”) in an approximately spherical shape. Another example of a radially progressive pattern excises a bore-hole about all the way through the tissue target (which may be alternatively considered a limited form of a longitudinally progressive pattern), then works up and down the interior surface of the bore hole to excise in successive radially expanded cylinders, approximately in a cylindrical or barrel shape, or a portion thereof.

A third type of pattern excises a section—for example a slab- or wedge-like section having a long axis approximately parallel to the longitudinal direction of advance of the cannula and surgical tool. The sections are excised, in some embodiments, from the center outward, along substantially a full longitudinal extent of the targeted tissue region. A potential advantage of excising a series of slabs or wedges of this type is that the excising tool remains roughly within a plane as it works. This can be helpful for monitoring using a planar section imaging method such as planar ultrasound, since it allows imaged features of the tissue to remain in plane during excision, which can assist in image interpretation. The slabs/wedges are optionally, but not necessarily excised in strict circumferential progression. Alternatively, for example, they may first excise in a star-shaped pattern that leaves some tissue intact between different excised regions, before that tissue too is excised. This a way of postponing the removal of supporting tissue, which potentially helps to reduce tissue motion throughout a longer portion of the earlier procedure.

It may be noted that although the bore hole may be excised up to or nearly up to the edge of the targeted tissue volume, excising it as a first step potentially reduces chances of passing into non-targeted tissue, since a maximum of surrounding tissue still remains at this early stage of excision.

Cutting of shapes like irregular and/or interrupted layers, slabs, or wedges may be accommodated by withdrawing and re-advancing the surgical tool as necessary to avoid certain areas while excising into others. Some volume shapes may be preferably excised using more than one pattern. For example, a blood vessel crossing within a tissue target (which needs to be avoided) could create a “shadow” which interferes with the excising of tissue behind it differently depending on whether the excising pattern is a layer pattern or a barrel pattern. Excising using both patterns could allow access to the excision of more tissue than either pattern alone.

A potential advantage of progressive pattern excision is that a pattern can be defined generically which is adaptable to a wide range of volumes (albeit there may be volume shapes which are incompatible with the robotic mechanics). A pattern which is partially progressive and partially saltatory (i.e., “jumping” to excise non-adjacent layers, slabs, and/or wedges of tissue) can also be predefined and is also so-adaptable. At the same time, the pattern may be defined to itself guarantee that movement through the target volume remains consistent with the mechanical constraints of the excising device for example, the pattern can guarantee that the positioning armature of a robotic device can move the excising end without concern for interference from surrounding tissue. Furthermore, the use of a consistent pattern potentially assists an observing operator to understand how tissue movements may be affecting the relationship of the working volume and the target volume.

Moreover, the pattern itself may be performed in an “open loop”, within a working volume that is precisely controlled even without continuous feedback as to where the robotic excision device is relative to boundaries of the target volume and/or a safe envelope of movement for operating in. Insofar as there is feedback, it is non-continuous, e.g., acting as a stopping exception. The pattern parameters can be defined so that an initial excision plan remains within an expected safe region. Alternatively, in some excision plan embodiments, the working volume is larger, e.g., equivalent to the volume of tissue which is targeted for destruction by the excision plan.

In either case, when the mismatch and/or risk of mismatch gets too high (e.g., as observed by a device operator, as set by a predetermined amount of excision operation, or another criterion), operation can be stopped. The excision plan may be changed, and excision according to a new pattern variant begun. Alternatively, operation can be restarted.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Tissue Movements (Brain Shifts)

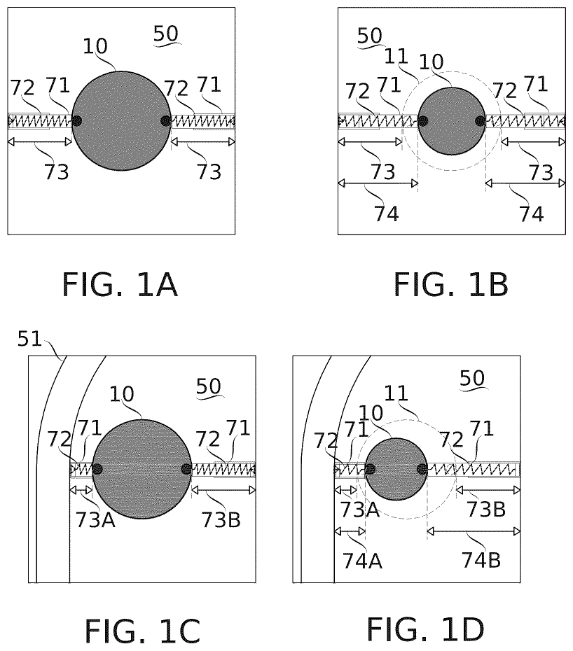

Reference is now made to FIGS. 1A-1D, which schematically represent a model of internal forces within a soft tissue (e.g., brain tissue), according to some embodiments of the present disclosure. FIGS. 1A-1B represent a case of isometric forces within uniform surroundings; Figures IC-ID represent an example of non-isometric forces within asymmetric surroundings. Changes in internal tissue forces (e.g., due to treatment, and in particular, treatment that results in internal pressure changes) may result in movements of that and/or surrounding tissue. In the case of brain tissue, such movement is also referred to herein as a “brain shift”.

In some embodiments of the present disclosure, a region of tissue 50 within which treatment is to be administered is considered as having internal pressure. This corresponds, for example, to the situation of brain tissue under compression maintained, e.g., by fluid pressure (blood and/or cerebrospinal pressure) within the confines of the cranium. In FIGS. 1C-1D, shell layer 51 is a less compressible layer, and may comprise, for example, cranial bone.

Accordingly, a given tissue portion 10 within the region of tissue 50 may be modeled as being connected from its surface to a position in surrounding space via springs 72 under compression. The compression may be measured, for example, with reference to atmospheric pressure; e.g., in a range of about 7-15 mm Hg above atmospheric pressure. Herein pressures referred to are pressures relative to atmospheric pressure, e.g., negative when lower than atmospheric pressure, and positive when higher.

Upon a change to the compressibility and/or internal pressure of tissue portion 10 (induced, for example, by removal of tissue from within), the equilibrium size of tissue portion 10 shrinks from the size of initial perimeter 11, to the size shown in FIG. 1B (e.g., lengthening, compared to FIG. 1A springs 72 from length 73 to length 74), or FIG. 1D (e.g., lengthening, compared to FIG. 1C, springs 72 from lengths 73A, 73B to lengths 74A, 74B).

When forces and surroundings are substantially uniform, tissue portion 10 may “compress in place” (e.g., to a fixed center). Asymmetries in the surroundings may alternatively induces a combination of compression and translation.

Optionally, the model includes dashpots 71, which slow the expansion of springs 71, and set a finite duration of time (of, e.g., seconds to minutes) over which shrinkage of tissue portion 10 occurs.

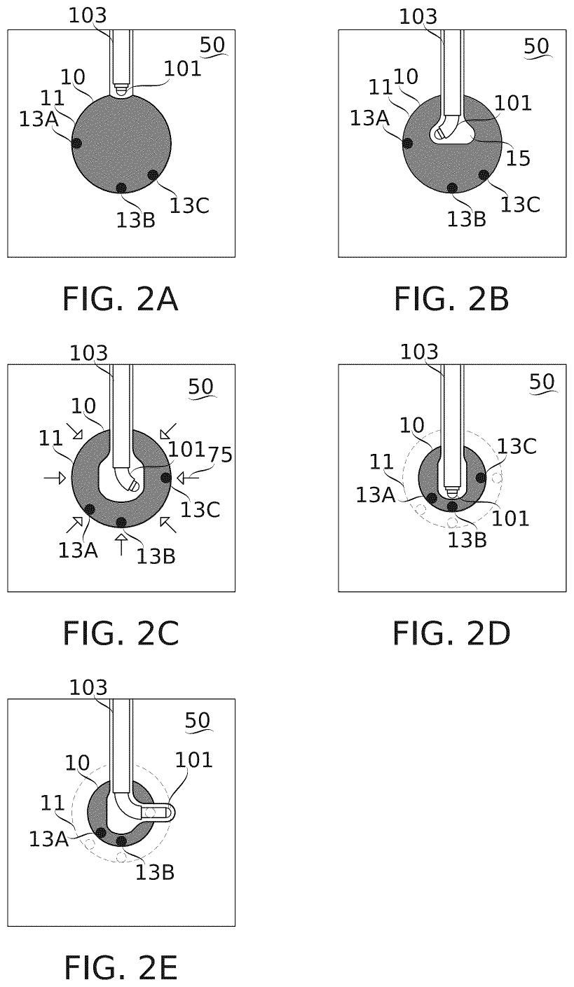

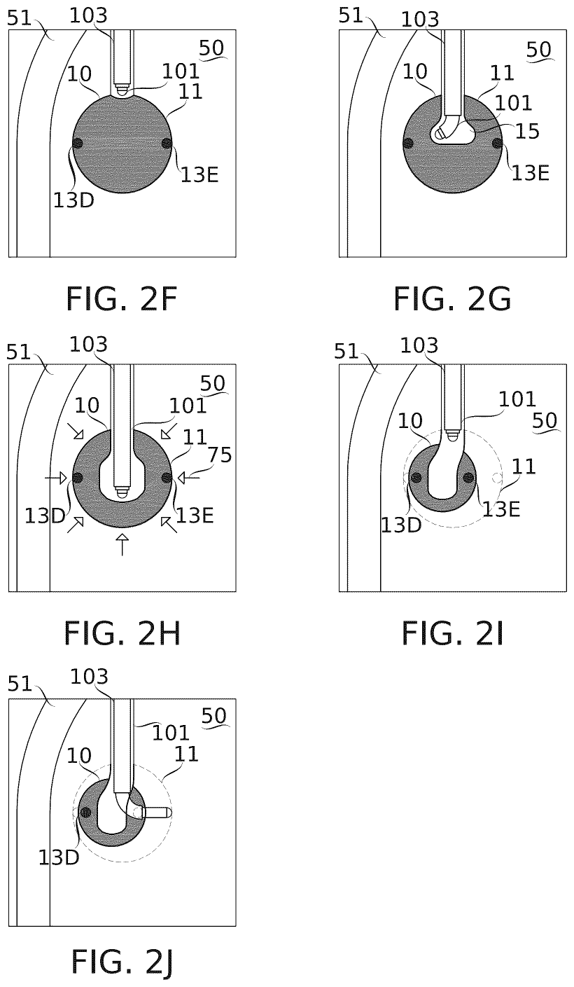

Reference is now made to FIGS. 2A-2J, which represent movements of tissue portion 10 under compression in response to hollowing-out, according to some embodiments of the present disclosure. FIGS. 2A-2E represent a case of isometric forces within uniform surroundings; FIGS. 2F-2J represent an example of non-isometric forces within asymmetric surroundings, e.g., surroundings near a less compressible shell layer 51 comprising, for example, cranial bone.

In some embodiments, the form of treatment which induces change to the compressibility of tissue portion 10 comprises removal of material from within tissue portion 10. This may occur, for example, in the removal of tumorous tissue, and/or removal of an epileptic focus in brain tissue.

FIGS. 2A and 2F represent an early phase of a medical procedure, wherein tissue portion 10 is to be excised from within tissue region 50. The size of tissue portion 10 shown also, in some embodiments, defines a reference working volume for a surgical tool 101, defined, e.g., by perimeter 11. Surgical tool 101 is positioned to be introduced into tissue portion 10 through cannula 103. Within tissue portion 10 are also marked sub-regions 13A, 13B, 13C (FIGS. 2A) and 13D, 13E (FIG. 2F), for reference.

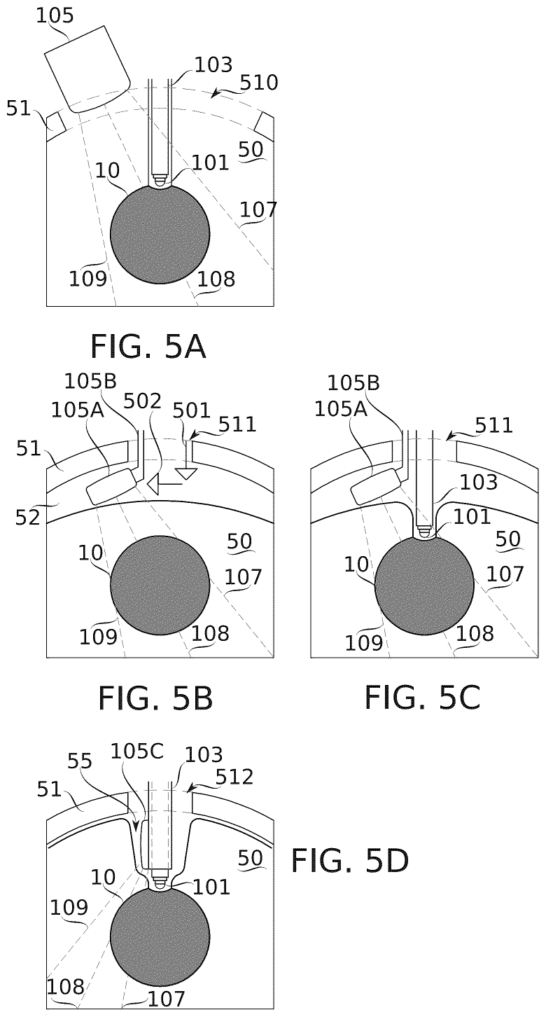

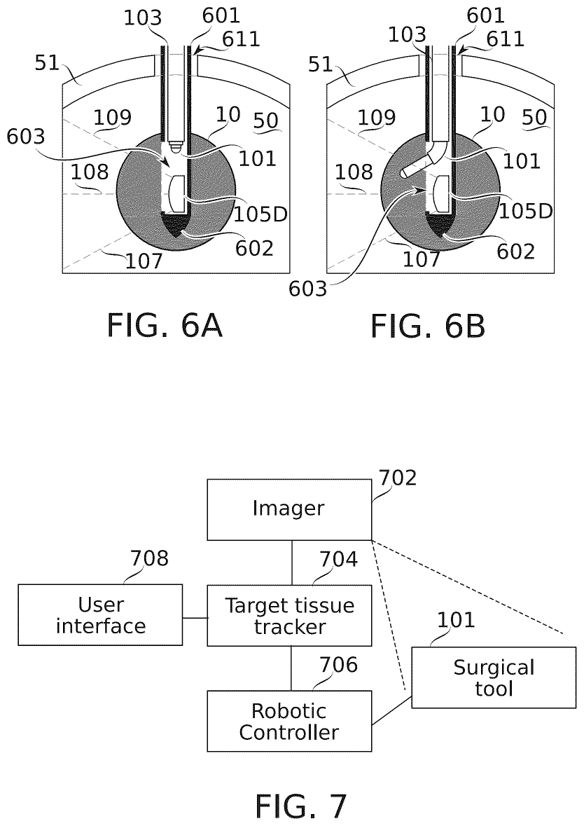

In some embodiments, surgical tool 101 comprises a longitudinally extended member having a flexible and steerable tip with an excising end. Surgical tool 101 is introduced into its working zone by distal advance, e.g., through a cannula 103. Movements setting insertion depth, rotation, and angulation of the flexible tip are actuated under the control of a robotic controller. Preferably, surgical tool 101 is configured (e.g., is stiff enough) to follow commanded movements faithfully, so that the commanded motion can be assumed to be the actual motion within the tolerances permitted by the procedure (e.g., within about 1 mm, 0.5 mm, or another tolerance). Optionally, imaging is used to provide correction feedback on at least one of the degrees of freedom of the surgical tool 101—for example, using one of the imaging head arrangements described in relation to FIGS. 5A-6B. In some embodiments, surgical tool 101 is an excision tool (e.g., useful to remove a tumor and/or epileptic focus). Surgical tool 101 is configured and operated to excise gradually, which potentially helps ensure that forces exerted on surgical tool 101 do not induce distortion that could create mismatch between actual movements and commended movements.

In FIGS. 2B and 2G, a cavity 15 has been initiated within tissue portion 10 by operation of surgical tool 101. For example, tissue previously part of tissue portion 10 has been macerated, flushed, and/or aspirated. Optionally, cavity 15 is generated by rotational movements of surgical tool 101 within tissue portion 10, so that cavity 15 approximates a rotationally symmetric hollowed-out volume. Optionally, pressure within initial cavity 15 reduces to a lower value than the static pressure within tissue region 50, e.g., to about atmospheric pressure.

Change in size of tissue portion 10 to a new equilibrium volume is not necessarily immediate in response to volume/pressure changes; and not necessarily an easily predicted (e.g., linear) function of the volume removed. At least initially, there may be structural integrity offered by the remaining volume of tissue portion 10 (and/or surrounding tissue region 50 itself) sufficient to at least partially resist collapse under the new imbalance of pressures. Additionally or alternatively, there may be a resistance to remodeling which acts as the dashpot 72 of FIGS. 1A-1D, slowing the change in size.

For example, the situations of FIGS. 2C and 2H represent an increased size of cavity 15, weakening tissue portion 10 sufficiently that it can undergo significant collapse. A pressure differential represented by arrows 75 (FIGS. 2C and 2H) leads, over time, to collapse of tissue portion 10 from its initial perimeter 11 (FIGS. 2D and 2I). In the case of FIG. 2I, there is also shown translational movement of tissue portion 10, due to the exertion of asymmetrical forces, e.g., as described in relation to FIGS. 1C-1D.

It should be noted that collapse potentially occurs, at least in part, before surgical tool 101 has finished excavating the material of tissue portion 10 which is targeted for removal. Thus, for example, if surgical tool 101 is moved to target removal of sub-region 13C or 13E based on initial location (e.g., as determined from pre-operative imaging), it will overshoot (FIGS. 2E and 2J), and intrude into an area of tissue region 50 which was not initially within the reference working volume of surgical tool 101 defined by perimeter 11. Particularly in structurally complex and/or regionally specialized organs such as the brain, this could result in damage with significant and potentially catastrophic consequences for patient outcome.

It is an object of some embodiments of the present disclosure to provide systems and methods for adjusting a reference working volume defined by the initial position of a tissue portion 10 to an actual working volume (optionally dynamically adjusted) which prevents or reduces overshoot, e.g., of the type described in relation to FIGS. 2A-2J. Potentially, this reduces damage to non-targeted tissue.

### Guided Adjustment of Working Volume of a Robotic Surgical Tool

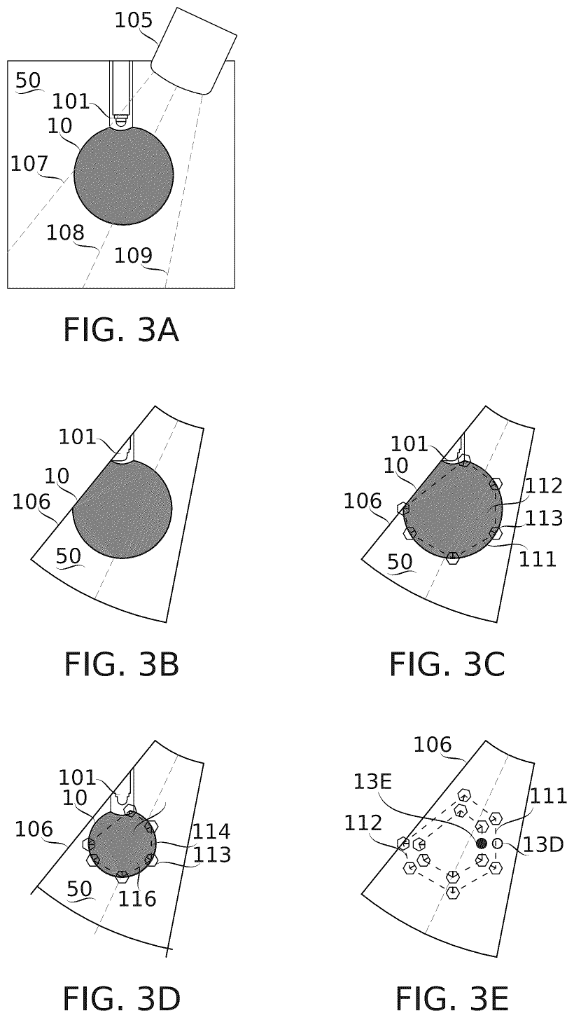

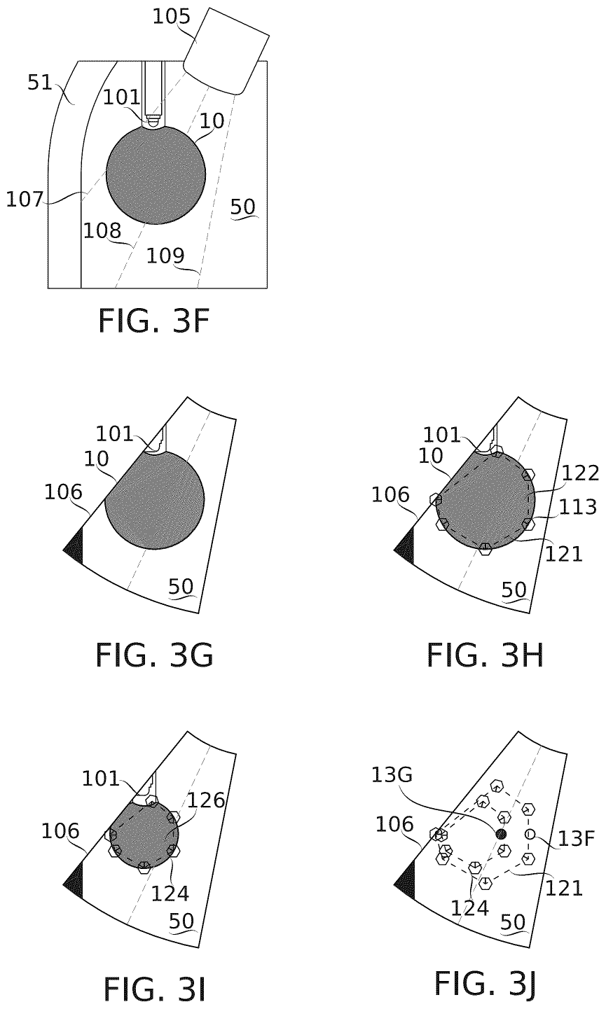

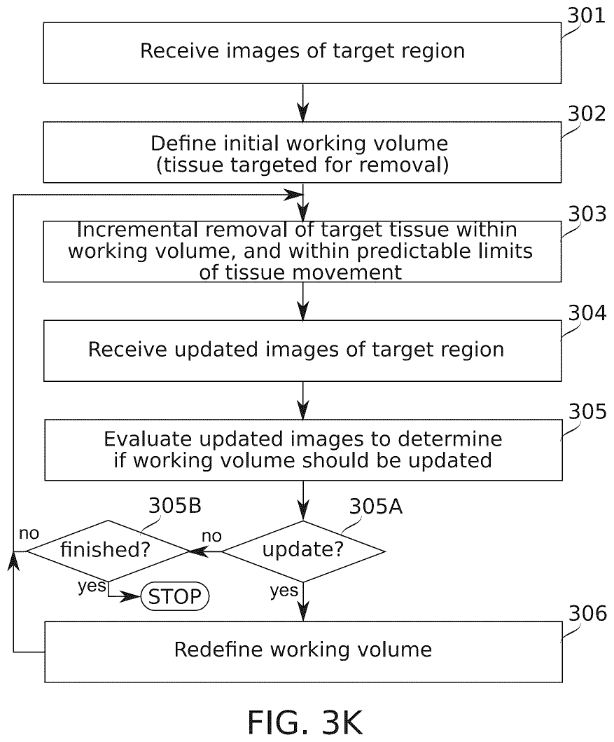

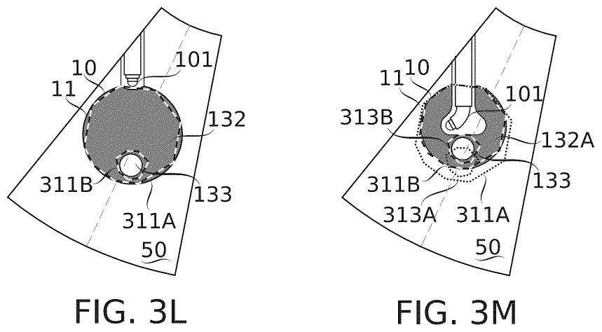

Reference is now made to FIGS. 3A-3J, which schematically represent a method of image-guided adjustment of a working volume 112 for a robotic surgical tool 101, according to some embodiments of the present disclosure. FIGS. 3A-3E represent a case of isometric forces within uniform surroundings; FIGS. 3F-3J represent an example of non-isometric forces within asymmetric surroundings, e.g., surroundings near a less compressible shell layer 51 comprising, for example, cranial bone. Reference is also made to FIG. 3K, which is a flowchart of a method of image-guided adjustment of working volume 112 optionally corresponding to what is illustrated in FIGS. 3A-3J, according to some embodiments of the present disclosure.

In the examples shown (FIGS. 3A and 3F), tissue portion 10 is targeted for removal by tool 101. Imager head 105 (which may comprise, for example, an ultrasound imaging device) is configured to image one or more planar sections 106 passing through tissue portion 10, e.g., a planar section within the viewing area defined by centerline 108 and outer boundaries 107, 109. The examples of FIGS. 3A-3J each illustrate just a single planar section; however, in some embodiments, a plurality of planar sections are imaged in order to define the extent of tissue portion 10 within three-dimensional space.

At block 301 (FIG. 3K), in some embodiments, one or more images are received (FIGS. 3B and 3G) of the region of tissue portion 10. The received images preferably comprise images obtained preoperatively, e.g., using an imaging tool (MRI and/or PET, for example) which is suited to high-contrast delineation of the mass which is to be excised. This potentially supports precise positioning of surgical tool 101 with respect to targeted tissue portion 10. Moreover, it may provide a precise initial determination of the boundaries of tissue portion 10 which is to be excised.

In some embodiments, one or more images including at least parts of tissue portion 10 are obtained intraoperatively, but preferably before actions which may promote brain shift. In some embodiments, these images are obtained using imager 702 (FIG. 7) which may be, for example, an ultrasound transducer. These images may be registered to the preoperatively obtained images, and/or to the position of the initial working volume (defined as described in relation to block 302). This provides, in some embodiments, baseline images which allow direct comparison of the initial state of targeted tissue portion 10 to later-obtained images of tissue portion 10. The images are preferably obtained in a plurality of planar sections 106 (e.g., several mutually offset parallel planar sections, and/or planar sections crossing through tissue portion 10 at different angles), to allow definition of an initial working volume 112 that takes into account details of the 3-D shape of tissue portion 10.

At block 302, in some embodiments, an initial working volume 112 is defined, e.g., as the region within a 3-D selection perimeter.

Optionally, the 3-D selection perimeter is based on automatic and/or manual segmentation of 3-D preoperative images received in block 301. In some embodiments, 2-D selection perimeters 111, 121 (FIGS. 3C and 3H) are calculated from the 3-D selection perimeter. This is a potential advantage for cases when pre-operative imaging provides higher resolution and/or contrast compared to imaging methods available intraoperatively.

Alternatively, a 3-D selection perimeter is reconstructed from one or more 2-D selection perimeters 111, 121. Initial working volume 112, 122 may be defined (e.g., when tissue portion 10 is known to be substantially spherical in shape) as the solid of rotation of a single 2-D selection perimeter 111, 121. The selection perimeters 111, 121 themselves are optionally defined manually, e.g., using a pointer to indicate anchor points 113 of selection perimeter 111, 121. Optionally, selection perimeters 111, 121 are defined at least partially automatically, e.g., using standard image segmentation techniques applied to 2-D images. Preferably, a plurality of 2-D selection perimeters 111, 121 are defined from a corresponding plurality of images in different planar sections 106, and combined to define working volume 112, 122.

In some embodiments, 2-D selection perimeters 111, 121 are defined by combined use of 2-D and 3-D imaging, e.g., by a method using both 2-D and 3-D imaging results to mutually constrain the shape and/or position of the initial working volume 112, 122.

However defined, each 2-D selection perimeter 111, 121 corresponds to (and is registered to) an imaging planar section 106, comprising a boundary region of tissue portion 10. In intraoperative images corresponding to each planar section 106, the corresponding boundary region may or may not be unambiguously distinct. A corresponding 2-D selection perimeter 111, 121 registered to the intraoperative image supplies missing boundary location information, if any.

It should be understood that surgical setup which accompanies the method of FIG. 3K is performed so that known movements of surgical tool 101 will be in known correspondence to the position of working volume 112, 122. Setup is optionally based on pre-operative images which constrain the placement of supports for cannula 103 and/or surgical tool 101 to a stereotactically precise location (e.g., a location defined within 1 mm, 100 μm, or another distance). Optionally, surgical setup combines use of both 3-D and 2-D imaging. For example, at least partial 2-D perimeters (like 2-D perimeters 111, 121) defined within two crossing image planes can be used to constrain and/or validate the positioning of a separately generated 3-D boundary. Even a single 2-D perimeter 111, 122 may be sufficient for registration of a 3-D boundary, e.g., if available scaling, offset and/or orientation information is of sufficient accuracy across the different image data sets.

At block 303, in some embodiments, removal of tissue portion 10 begins (or, in later iterations, continues). The removal is performed automatically (e.g., by robotic manipulation). Initially, removal is limited by the bounds of working volume 112, 122; in later iterations, removal is limited by the bounds of working volume 116, 126.

At least one later time (block 304), one or more new images are taken. In some embodiments, imaging takes place at predetermined times and/or waypoints during the procedure. Examples of such waypoints include after some fraction of tissue removal by volume has been performed (e.g., about 50%, about 75%, or another fraction). Times may be, e.g., after every one, two, three or more sweep rotations of the excision tool through the working volume; or after fixed intervals of time (e.g., every 30 seconds, every minute, or another interval). Optionally or additionally, new images are obtained at the discretion of a device operator.

New images may comprise images sufficient for partial or complete characterization of the procedure progress. For example, images may be continuously (e.g., at several times per second) taken for a single cross-section of the target tissue region; and when it appears that tissue movement and/or procedure progress may necessitate adjustment to the working volume, a more complete set of images (e.g., from a plurality of directions) may be obtained to be used in selecting new surgical parameters.

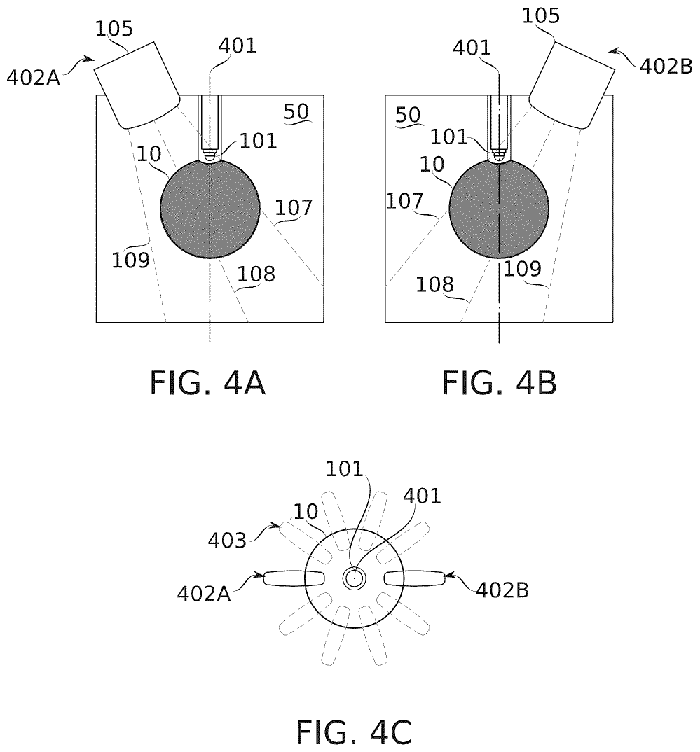

Systems and methods of imaging are further described, for example, in relation to FIGS. 4A-6B.

Optionally, at block 305, the images are evaluated for whether or not a new working volume should be defined. The evaluation is optional, for example, since it may be predetermined (e.g., based on milestone) that a new working volume is to be defined, and/or because a computerized system controlling the procedure is configured to automatically recalculate the working volume as the procedure progresses. Optionally, evaluation comprises inspection of images by a device operator who determines that the target tissue portion 10 is now so changed from its original position and/or size (and/or the progress of removal is sufficiently advanced) that the working volume of surgical tool 101 should be adjusted to fit newer conditions. Optionally, evaluation comprises an automatically performed assessment that the position of surgical tool 101 is at risk of exceeding the bounds of targeted tissue portion 10. The evaluation is optionally based, for example, on image segmentation results. Optionally, the evaluation uses tracks and/or limits of tissue removals already performed during the procedure as fiducial marks. Optionally, the movement of image feature patterns in the vicinity of earlier defined perimeters of the targeted tissue region 10 (even if not themselves part of the perimeter) are used to assist in the evaluation of tissue movements. Additionally or alternatively, the size and/or position of the void of removed tissue is measured, and its shrinkage and/or movement used as an indicator of the movement of targeted tissue region 10 overall.

At block 305A, in some embodiments, the flowchart branches according to whether or not the working volume 116, 126 is to be updated. If there is no current decision to update the working volume (and if tissue removal is unfinished, per block 305B), then the flowchart returns to block 303.

Otherwise, a new working volume 116, 126 is to be defined. The method proceeds to perform this operation at block 306, in some embodiments.

In some embodiments, redefinition comprises definition of one or more new 2-D selection perimeters 114, 124 (e.g., using anchor points 115), using the new images. Taken together (e.g., taking into account the three dimensional arrangement of their respective images' imaging planes), the new 2-D selection perimeters 114, 124 define the new working volume 116, 126 which reflects the updated state of targeted tissue portion 10. In FIG. 3D, new working volume 116 is shrunken from original working volume 112. In FIG. 31, new working volume 126 is offset as well as shrunken from original working volume 122.

In some embodiments, the operation of block 306 is implemented by providing user interface elements that allow an operator to mark out new 2-D selection perimeters 114, 124 manually, e.g., as an overlay on images newly acquired at block 304. At least where there is sufficient image contrast between target tissue region 10 and surrounding tissue 50, standard image segmentation procedures (e.g., threshold-based, with additional smoothing/noise removal as necessary) are optionally performed to obtain the new working volume 116, 126 e.g., by automatically generating 2-D selection perimeters 114, 124 from a plurality of images. Optionally, image analysis methods described in relation to evaluation at block 303 are used for automatically determining modifications to the working volume; e.g., using tracks and/or limits of tissue removals already performed during the procedure as fiducial marks, and/or using the movement of image feature patterns in the vicinity of earlier defined perimeters of the targeted tissue region 10. Any automatic determination is optionally adjusted by manual inputs provided through a human-computer interface (user interface).

Until tissue portion 10 is sufficiently removed, blocks 303-306 repeat; then the method of FIG. 3K terminates. A “finished” decision is optionally taken at any stage of the flowchart (for example, as indicated for block 305B).

The cycles of working volume updating occur while a patient is undergoing the same surgical procedure; e.g., in the same surgical bed and/or while a mount which aligns surgical tool 101 to its target in the body of the patient remains in alignment. Typically, the cycles occur while the surgical tool 101 remains inside the body of the patient.

FIGS. 3E and 3J illustrate how updating the working volume helps with targeting. The targeted tissue initially located within sub-region 13D, 13F (near the bounds set by 2-D selection perimeter 111, 121) moves, during removal of nearby tissue, to sub-region 13E, 13G (near the bounds set by 2-D selection perimeter 114, 124). Changing the working volume (e.g., according to the 2-D selection perimeter) helps to prevent unintentional removal of tissue which is outside of targeted tissue portion 10.

Any of several variant operations are optionally operations of the method of FIG. 3K and the examples of FIGS. 3A-3J. These variant operations are changed, for example, with respect to aspects of: pattern of tissue removal, method of imaging, method of image evaluation, and/or method of working volume updating. Embodiments implementing some of these different approaches to these aspects are further described herein, by way of example.

While many patterns of tissue removal are possible, a particular feature characterizing some of these patterns relevant to tissue movement and working volume adjustment/monitoring is the extent to and manner in which the patterns are “surface first” or “center first” in order of tissue removal. Using a surgical tool 101, a single procedure will excise at least one surface portion first. This may be followed by excisions which are directed toward minimizing center excision on the way to reaching another outer surface (e.g., by boring directly to the surface distally opposite the entry location of the surgical tool 101); or, in contrast, excisions which are directed toward excavating the center while avoiding the outer surface. It should be understood that a range of excision patterns in-between these examples may result in more or less central volume removed per unit of surface area visited.

The example of FIGS. 2A-2E, for example, shows a case of primarily “center first” tissue removal, after initial penetration to a central location within targeted tissue portion 10. This pattern may eventually result in a tissue movement pattern which looks like global shrinkage, wherein the perimeter of tissue portion 10 may be modeled to constrict approximately by the same amount on all sides. Such a pattern of tissue removal has a potential advantage, insofar as it establishes a buffer of targeted tissue during early tissue removal that can act as a margin of safety. Especially in the early stages of tissue removal, there may be a tendency for bending and/or rotating movements of the surgical tool 101 within the confined space available to also move tissue, increasing the unpredictability of exactly what tissue is being excised at any given moment. Performing early removal from deep within the tissue helps keep this from resulting in errors wherein tissue not targeted for removal is inadvertently destroyed.

As the internal volume opens up, the working distal end of surgical tool 101 will tend to excise tissue at more distal portions of its tip as it rotates, and excise less often upon lateral aspects of the tip. Relatedly, there is opened up more room for bending and/or rotation of the surgical tool 101 potentially minimal displacement of surrounding tissue induced by such bending and/or rotation.

Potentially, working to predominately excise centrally first allows a relatively longer period of tissue removal to occur before mismatch between initial working volume and current target tissue region position threatens removal of non-targeted tissue, e.g., because the outer shell retains some level of structural integrity resisting collapse. This may have the potential advantage of reducing the time between reviews of the working volume's current accuracy, and/or the potential advantage of allowing the robotic system to safely work “blindly” for a longer period before new images must be obtained/evaluated to check on the working volume limits. However, to finally address excising at the edges of the volume, a significant adjustment to the working volume is likely to be needed.

Alternatively, in some embodiments, a pattern of tissue removal is more directed to earlier excision out to the surface of the targeted tissue portion 10. Some limits on this are imposed by the physical shapes that can be assumed by surgical tool 101 without displacing tissue by rotational and/or bending contacts along the shaft of the surgical tool 101. For example, it may be preferable, early in a procedure, to remove tissue from and between the proximal (“top”, as shown) and/or distal (“bottom”) boundaries of the targeted tissue portion 10, relative to the axis along which surgical tool advances. The outside boundary of tissue midway along the distal-proximal axis may be more difficult to access before the middle is fully cleared, but could be reached, e.g., by directly boring right up to it through a gradually advancing bend, retracting the surgical tool 101, rotating the surgical tool 101, then advancing again through a bend oriented in a new radial direction. This could be done in a pattern that leaves a relatively incompressible scaffolding of tissue in place for a longer period of time. Eventually the scaffolding too would be removed, and at this stage, compressibility within the target area could increase rapidly.

Surface-first patterns provide a potential advantage for allowing more sensitive edge regions to be excised up to (and perhaps also along) before the target tissue portion 10 is weakened enough that significant tissue movements develop. Working alternately on different sides/in different directions (e.g., in layers on the distal and proximal sides) may also help to equalize forces, minimizing displacement (e.g., of the central regions of the targeted tissue portion 10) until it is time to remove them as well. Insofar as movements of the excising surgical tool 101 are robotically controlled, in some embodiments, the complexity of such a movement pattern is potentially acceptable. An excision method such as this has the potential advantage of allowing a longer initial working time before adjustments due to motion are needed, since some surface regions are excised early before movements are likely to have developed, while the center can still be excised safely later on even if some movement occurs. The difference will depend, e.g., on how compressible a certain “scaffold” pattern (comprising intact tissue portions which cross through an excised void) is compared to a more “shell”-like pattern. However, it should be kept in mind that the early excising out at the edges could still threaten to destroy non-target tissue, insofar as smaller movements and/or errors, if they do occur, can result in more meaningful unintended effects.

It may be understood, in summary, that concentrating excisions centrally potentially allows a greater safety margin in removing an initial bulk of material, greater simplicity in excision pattern, and/or more working time before new imaging/boundary verification is needed; but it will be very likely that edges of the working volume will move (and optionally need to be re-identified) before they begin to be excised.

Concentrating at least some early excision work on surface locations potentially reduces the early safety margin (e.g., increasing the value of close monitoring throughout the procedure); but may, nevertheless, allow some of the working volume's boundary surface area to be excised before adjustments are actually required.

This can be an advantage, for example, insofar as pre-operative imaging may be available which is more accurate and/or definitive (e.g., higher-contrast) in identifying target tissue than whatever intraoperative imaging method is available. For example, selective tracer- or other property-based imaging (e.g., PET imaging, MRI imaging) may be available pre-operatively to delineate boundaries based on functional and/or biochemical markers; while intraoperative imaging may be limited to a technique relying on a potentially lower inherent contrast in how tissues of different types interact with radiant energy. Tracking boundaries may be harder in the latter case, so excising up to at least some portions of them may be preferable.

It may also be noted that insofar as earlier removal of tissue is patterned to include at least some excision up to the edges of the target region, it potentially creates an edge-identifying fiducial mark (the tissue boundary itself) which is higher contrast than the original tissue-tissue boundary. This in turn may assist manual and/or automatic interpretation of intraoperative imaging performed to identify where the remaining uncut edges are located.

Even without excising up to the edge, the boundaries of any hollows created within the target region are similarly potential fiducial marks, albeit with a more complex relationship to the outer margins of the target region. For example, the edge of a hollow excised to within 3 mm of the outer boundary of the target region is expected to afterwards shift at most insignificantly from a 3 mm distance from the outer boundary (as long as it remains uncut); insofar as any residual overall movements may be assumed to be mostly be due to redistributions of force and changes in shape outside the target tissue area. This assumption works for the simple case of there being no significant shift until after a particular partial excision has been made, e.g., for a boundary established early in excision. Optionally, excised volume changing and/or shifting is monitored over time (e.g., by intraoperative imaging), and this time course used to correct the excision expected from commanded movements alone into an estimated excision volume (and associated boundary). The current boundary of the targeted tissue portion 10 can then be calculated by applying an appropriate offset from the estimated excision volume.

It is noted that, e.g., for a target region comprising an artery of sufficient significance, an excision pattern might be designed to leave a central region unexcised; in this case, the central region also defines an “outer boundary” of the target tissue portion for purposes of maintaining avoidance.

In terms of the method of FIG. 3K, these considerations may also differentially impact when and/or how often the loop of blocks 303-306 should repeat. More particularly, excising in regions of greater risk (e.g., near the edges and/or near large blood vessels) is preferably accompanied by more frequent (optionally continuous), imaging and evaluation (blocks 304-305) of where the excision tip of surgical tool 101 is relative to the edges of targeted tissue portion 10.

Reference is now made to FIGS. 3L-3M, which schematically represent adjustment of a working volume 132 comprising a blood vessel 133 for a robotic surgical tool 101, according to some embodiments of the present disclosure.

In some embodiments, targeted tissue comprises a structure which should be avoided, such as a blood vessel 133. Working volume 132 is optionally specified (FIG. 3L) with boundaries comprising a plurality of volumes, e.g., the area within boundary 311A, excluding the area within boundary 311B (bounds are shown for just one plane; in some embodiments, bounds are defined within different planes and merged to a 3-D definition of working volume 132).

After partial excision and collapse of tissue within working volume 132 (FIG. 3M), new bounds 313A, 313B (defining new working volume 132A) may be defined instead. It should be noted that blood vessel 133 may “shadow” some regions of the tissue within working volume 132, 132A—that is, surgical tool 101 may not be able to access all designated regions without passing into the region of blood vessel boundaries 311B and/or 313B. Optionally, such cases result in unexcised areas.

### Imaging Arrangements

Reference is now made to FIGS. 4A-4C, which schematically represent an imaging pattern which may be used to monitor tissue excision, according to some embodiments of the present disclosure. In some embodiments, the monitoring is performed to support operations of FIG. 3K, e.g., to provide the images of block 304.

In each of FIGS. 4A-4B, a cross-section of tissue portion 10 within a region of tissue 50 is imaged from imager head 105. The viewing area of the image is defined by centerline 108 and outer boundaries 107, 109. In FIG. 4C, the head positions 402A, 402B are indicated as seen from a viewing angle positioned along axis 401 (e.g., axially above a longitudinal axis of surgical tool 101). A plurality of other imaging positions 403 are shown in dotted lines radially arranged around axis 401.

In some embodiments, a three-dimensional shape of tissue portion 10 is reconstructed from a plurality of images obtained from a plurality of imaging positions 402A, 402B and/or 403, corresponding to images through a plurality of different intersecting planes of tissue portion 10. Optionally, definition of selection perimeters is likewise performed within a plurality of images.

Reference is now made to FIG. 5A, which schematically illustrates a large-access configuration for imaging a target tissue portion 10 using an imager head 105, according to some embodiments of the present disclosure. Optionally, imager head 105 comprises an ultrasound transducer. In some embodiments of the present disclosure, a sufficient quality of ultrasound images used to guide a procedure is achieved by avoiding imaging through bone (e.g., in cases where shell 51 comprises cranial bone). Using a large-access approach, an imager head 105 can be placed within and near the edge of an access hole cut into bone, and oriented so that its imaging field (defined, e.g., between lines 107 and 109 and centered on centerline 108) includes at least a partial cross-section of target tissue portion 10. Different image planes are selected by moving imager head 105 around the perimeter of access aperture 510. Cutting such a large cranial hole is, however, a potential disadvantage insofar as it is more invasive; representing, e.g. increased risks for surgical safety and recovery.

Reference is now made to FIGS. 5B-5C, which schematically illustrate a reduced-size access configuration for imaging a target tissue portion 10 using an imager head 105A, according to some embodiments of the present disclosure. In some embodiments, imager head 105A is configured to insert through aperture 511 into a sub-cranial space 52. Optionally, imager head 105A (comprising, for example, an ultrasound transducer) is attached to an angled mount 105B, and is inserted by passing first downward through aperture 511 (e.g., along arrow 501), after which it is rotated and/or translated (e.g., along arrow 502) to a sub-cranial position underneath bone. In some embodiments, anther sequence of movements is used; for example, tilting angled mount 105B so that imager head 105A passes into aperture 511 narrow-side first, and then rotating angled mount 105B to finish “hooking” imager head 105A underneath cranial bone. In some embodiments, surgical tool 101 is introduced after imager head 105A is in place. The angled mount 105B, in some embodiments, is itself rotatingly coupled to the surgical tool 101 (e.g., via a bracket attached to a shaft of the angled mount), allowing it to be rotated around a longitudinal axis of surgical tool 101.

Reference is now made to FIG. 5D, which schematically illustrate a cannula-mounted imager head 105C, according to some embodiments of the present disclosure. In some embodiments, an imager head 105C (comprising, for example, an ultrasound transducer) is attached to an external surface of cannula 103, and configured to image at a steep downward angle, for example an imaging cross-section as indicated by lines 107, 109 on either side of centerline 108. This potentially allows maintaining a smaller size of aperture 512 used for intracranial access, while still placing the imager head 105C in a position that avoids imaging interference from bone tissue. It is noted in particular that an aperture 512 used with the configuration shown in FIG. 5D is not necessarily radially symmetric. For example, aperture 512 may be excised with a first internal area which is sized for passage of cannula 103, and a second, radially offset area, which is sized for passage of imager head 105C. Once imager head 105C is inserted past the aperture, it can rotate against soft tissue. This configuration is potentially most advantageous when surgical tool 101 is accessing targeted tissue portion 10 via a pre-existing sulcus 55.

Reference is now made to FIGS. 6A-6B, which schematically represents a cannula-mounted imager head 105D, according to some embodiments of the present disclosure. In the configuration shown, cannula 103 is supplemented by an imaging cannula 601, having an interior large enough to accept cannula 103 within it. Imaging cannula 601 comprises imaging head 105D, comprising, for example, an ultrasound transducer. During use, a distal end of imaging cannula 601 including imaging head 105D is optionally positioned (e.g, via insertion through aperture 611) within target tissue portion 10, allowing “inside out” imaging to be performed, e.g., along a planar region delimited by lines 107, 109 and having centerline 108. In some embodiments, imaging head 105D is fixedly attached to an inner lumenal surface of imaging cannula 601, and rotating imaging cannula 601 around its long axis also rotates the image plane of imaging head 105D.

A potential advantage of this placement of the imaging head 105D is allowing a particularly small access aperture 611 to be used (e.g., having a diameter down to near the outer diameter of imaging cannula 601 itself.

In some embodiments, imaging cannula 601 comprises a laterally positioned window 603 sized to allow surgical tool 101 to pass out in order to work on tissue outside of imaging cannula 601. For example, surgical tool 101 may have an outer diameter of about 3 mm, and lateral positioned window 603 may be about the same size in a horizontal direction, plus enough tolerance to allow smooth passage. Longitudinally, window 603 extends longer (e.g., by another 5-10 mm), to allow a wider range of longitudinal movement of surgical tool 101 without moving cannula 101. Imager head 105D is optionally positioned along window 603, and oriented so that radiant energy (e.g., sound waves) can exit and/or enter cannula 601 through window 603. For example, the portion of window 603 extending along imager head 105D may be, for example, about 5 mm long. Optionally window 603 is divided into separate compartments, one for use by surgical tool 601, and one to form an aperture for imaging head 105D. It is a potential advantage to align the plane within which surgical tool 101 advances with a planar region imaged by imager head 105D, to allow direct (and optionally real-time) imaging of movements of surgical tool 101.

Optionally, outer cannula 601 comprises a sharpened tip 602 which assists in penetrating into tissue. Before insertion, a pathway for imaging cannula 601 is optionally first cleared using other means (e.g., using cannula 103 and surgical tool 101). Pre-insertion clearance optionally also comprises clearance of tissue within tissue portion 10 which otherwise would have access blocked by imaging cannula 601. It is noted that window 103 is optionally withdrawn to allow increased access to more superficial (proximal) portions of tissue region 10; however, it may be long enough toward a proximal direction that it ordinarily does not interfere with lateral access proximally.

### Surgical Treatment Systems

Reference is now made to FIG. 7, which schematically illustrates a system for target-adjustable treatment of tissue by a robotically operated surgical tool, according to some embodiments of the present disclosure.

The block labeled surgical tool 701 corresponds, in some embodiments, to surgical tool 101, for example as described in relation to FIGS. 2A and 2F.

Robotic controller 706 is configured to direct the movements of surgical tool 101. In some embodiments, robotic controller 706 operates autonomously to select motions of surgical tool 101, working within a set of parameters describing the current target. Robotic controller 706 may interrupt activity to allow resetting its operating parameters. The interruption is optionally automatically triggered or manually triggered. An automatic interruption trigger may be based, for example, on elapsed procedure time, fractional procedure progress, and/or estimation of target tissue movement (e.g., modeled and/or detected, for example by imaging). Optionally, operating parameters are reset without interruption of movement.

In some embodiments, robotic controller 706 receives new operating parameters (particular relating to target and/or safe envelope of movement positioning) from target tissue tracker 704. Target tissue tracker 704 is configured to define and as needed update the working volume within which surgical tool 101 operates, for example as described in relation to FIG. 3K.

In some embodiments, target tissue tracker 704 in turn receives tracking data from an imager 702, configured to monitor the state of tissue in the region of surgical tool 101. Optionally, user interface 708 (comprising, for example, a display, keyboard, touch panel, mouse, and/or other human/computer interface devices) is operated to control, confirm, and/or assist operation of tissue tracker 704.

### Summary of Guided Adjustment of Working Volume of a Robotic Surgical Tool

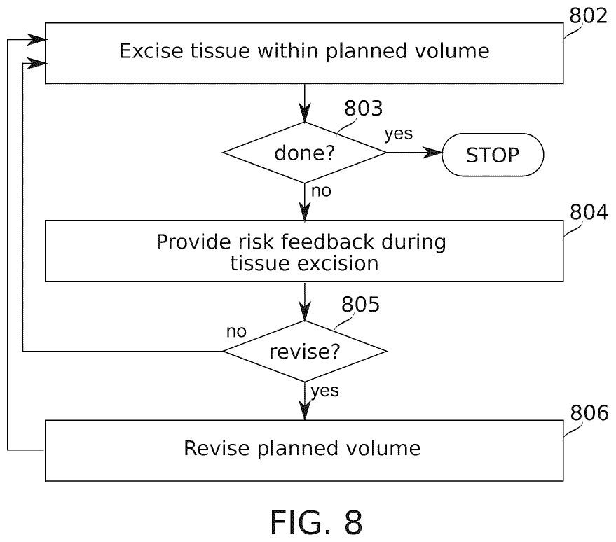

Reference is now made to FIG. 8, which is a flowchart of a method of risk-guided excision of tissue, according to some embodiments of the present disclosure.

At block 802, in some embodiments, tissue is excised within a planned volume, which is a working volume that is provisionally considered safe for excision activities. The working volume remains fixed in position until revised (block 806). The working volume may comprise all or a part of a region of tissue targeted for excision.

Meanwhile, at block 804, in some embodiments, risk feedback is provided periodically during tissue excision as long as tissue excision overall remains incomplete (e.g., as assessed at block 803).

Risk feedback may comprise any indication that allows a judgement of risk that continued excision within the planned volume could cause excision of non-targeted tissue. The risk in particular comprises risk that tissue has shifted in the vicinity of the planned volume so that non-targeted tissue has actually come into the planned volume, and/or is approaching it.

Risk feedback, in some embodiments, comprises display to a user of an image of a current and/or recent state of tissue within and around the working volume (e.g., imaged by ultrasound), and an at least partial indication of where the working volume is situated relative to tissue shown in the image. The user may review the image to judge if tissue shift engendering risk of non-targeted tissue excision has occurred and/or is occurring.

In some embodiments, the risk feedback comprises measuring of an elapsed time or progress of excision. In some embodiments, the risk feedback comprises an automatic (e.g., image processing based) judgement of brain shift. In some embodiments, risk feedback includes indication and/or assessment of direction of movement, magnitude of movement, non-targeted regions which appear to be most imperiled by continued excision, and/or non-targeted regions which are particularly critical to avoid.

At block 805, in some embodiments, a decision is made as to whether or not it is time to revised the planned volume, based on the provided risk feedback. If not, the flowchart continues with the excision and risk feedback providing of blocks 802 and 804.

Otherwise, at block 806, the planned volume is revised. This optionally comprises redefining outlines on 2-D images, or another boundary redefinition which accounts for shifts in the target region so that excision can resume within the revised planned volume with reduced risk.

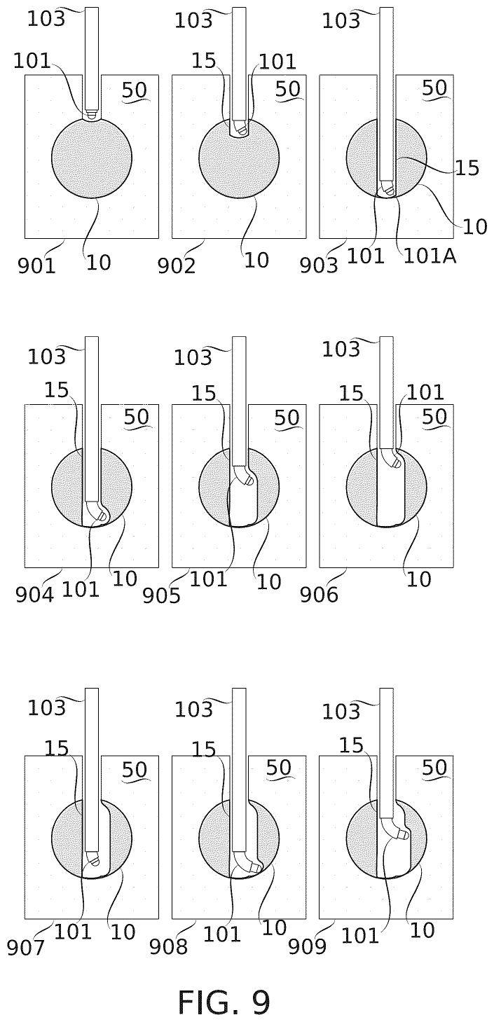

Reference is now made to FIG. 9, which schematically represents a time series of movement pattern of a cannula 103 and surgical tool 101 to excise a targeted tissue portion 10 from within a larger tissue region 50, according to some embodiments of the present disclosure. The time series is shown through the sequence of panels 901-909. Apart from the initial bore hole to establish the movement throughout the proximal-to-distal length of the working volume, the pattern shown in FIG. 9 is neither “surface first” nor “center first”, in the sense described in relation to FIGS. 2A-2E; instead, it proceeds by excising numerous sections, each from center to surface, for example as slabs or as wedges.

In the movement pattern shown, surgical tool 101 is first worked in a direction along the longitudinal axis of cannula 103, from one (proximal and more superficial) side of tissue portion 10 (as in panel 901) to the other, distal side (as in panel 903). While advancing, tool 101 is slightly protruded along a curving path from cannula 103, and rotated. The protrusion is enough so that the working distal end 101A is of surgical tool 101 positioned where it excises surrounding material to form the initial wall of cavity 15 at a position a small lateral distance outside the circumference of cannula 103. The distance is enough outside that circumference that cannula 103 can be advanced through the growing cavity 15. It is, however, minimally further, with the object of retaining structural integrity of the surrounding tissue. This potentially reduces freedom of the tissue to rearrange itself during this portion of the procedure.

If, as it rotates to excise tissue from the circumferential wall of cavity 15, the protruded configuration of surgical tool 101 does not also reach to excise the axial center of the cylindrical region of cavity 15, then working distal end 101A may be periodically withdrawn slightly to re-orient it to a position where it excises distally straight ahead of cannula 103.

It may be noted that the diameter of the borehole may be small enough that excision during proximal-to-distal movement can be monitored by a planar imaging method such as ultrasound, substantially from within a single plane containing the axial extent of the bore hole. The “out of plane” region, if it exists, is small enough that changes in the imaging plane can be relied on for feedback.

Next, as illustrated in panel 904, cannula 103 is slightly withdrawn and surgical tool 101 protruded slightly further, so that it reaches a little further laterally. By the end of the withdrawing motion (panel 906), surgical tool has, accordingly, excised a further layer of target tissue portion 10 from one side of cannula 103.

In some embodiments, surgical tool 101 is not rotated during excision, or rotated very little (for example, “wiggled” a few degrees back and forth) so that it excises away a slab-shaped thickness of tissue about as wide as the working distal end 101A. Lack of rotation means that the tool may stay at least roughly within an imaging plane.

Whether or not the imaging plane moves together with rotation of the surgical tool 100, this is a potential advantage, insofar as it means that a single plane can be monitored for a continuous period of time while it is being cleared.

Tracking progress and tissue repositioning from ultrasound images can use not only identification of static features (such as image contrast generated at boundaries), but optionally also of identifying movements (individually or in coordination) of low contrast and/or interrupted contrast features which may be difficult to interpret when static. Accordingly, a constantly moving image plane could interfere with feature tracking, since features going in and out of the image plane would appear and disappear from view, and may be difficult to re-identify between their appearances. A planar excision pattern can allow the image plane to remain static for a longer period of time.

Alternatively, in some embodiments, surgical tool 100 is rotated by a significant amount as it is withdrawn; for example, a half arc, quarter arc, or eighth arc. This may give up some of the potential advantages of continuously tracking within a single imaging plane just described.

It should be noted, however, that the surgical tool 101 has a thickness, so a few degrees of movement may not necessitate repositioning of an imaging plane which monitors excision progress. The amount of rotation (in degrees) may be reduced with increasing radial distance from the cannula 103 which defines the axis of rotation, so that rather than excising a roughly triangular wedge, a slab with more nearly parallel sides is removed. In some embodiments, an ultrasound transducer coupled to surgical tool 101 so that it follows its rotational movements may be loosely coupled, so that it can remain in a fixed position while surgical tool 100 “wiggles” (e.g., rotates around the axis of cannula 103 by up to about 10°, 20° or 30°), but follows larger rotational movements to keep the surgical tool 101 in plane.

In one or more further excision passes (panels 907-909), additional layers of target tissue portion 10 may be excised, extending the section already begun. The further excision passes may begin with surgical tool 101 being placed first back on the distal side of the targeted tissue portion, and then excising the next layer during proximal movement of cannula 103 and surgical tool 101. Excising in this distal-to-proximal direction provides a potential advantage over proximal-to-distal for excising. For example, the tissue surface being excised at can be slightly stretched on one side (due to contact with the working distal end 101A), while inducing relatively less compression of tissue on the other side—since the upper (more proximal) side of surrounding tissue in target tissue region 50 is freer to move in response to forces than the lower (more distal) side, which is supported by deeper tissue structures. Apart from being potentially damaging, deformation of target tissue due to compression may make the process of excision less controlled.

Another potential advantage of this excision pattern is that cannula 103 can continue to support and stabilize positioning of the un-excised region of target tissue region 10 while completing excision of at least the first side of target tissue region 10. Tissue collapsing into cavity 15 is essentially constrained to collapse to the left (as shown in the panels), since cannula 103 blocks movement in the other direction. The resulting increase in predictability of tissue movement may reduce the frequency of the need to stop and reset the excision pattern to adjust for tissue movements.

Collapse may also be postponed in the proximal-to-distal direction, since proximal-to-distal tissue support remains intact out to about the middle of the targeted tissue portion 10 until, e.g., excision on the last section begins.

### General

It is expected that during the life of a patent maturing from this application many relevant robotic surgical tools will be developed; the scope of the term robotic surgical tool is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hearby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1. A method of guiding a robotically controlled surgical tool to excise a targeted tissue portion within neural tissue, the method comprising
receiving a first excision plan specifying a first working volume of the targeted tissue portion;
excising targeted tissue with the surgical tool with movements limited to within the first working volume according to the first excision plan; and
before the targeted tissue portion is fully excised:
imaging the targeted tissue portion in its incomplete excised state,
generating a second excision plan defining a second working volume, based on the imaging, and
continuing excision with the surgical tool according to the second excision plan, and while limited to the second working volume;
wherein generating the second excision plan comprises:
receiving an indication of a shifted boundary of the targeted tissue portion, and
defining the second working volume within the targeted tissue portion, based on the indication of the shifted boundary.

2. The method of claim 1, wherein the first and second excision plans specify automatic motions of the robotically controlled surgical tool within their respective first and second working volumes.

3. The method of claim 2, wherein the automatic motion of the surgical tool is performed according to a progressive cutting pattern.

4-5. (canceled)

6. The method of am claim 1, wherein the indication of a shifted boundary is derived from images obtained by the imaging.

7. The method of claim 6, wherein the images the second excision plan is based on indicate boundaries of the targeted tissue portion with adjacent tissue less distinctly than the pre-operative images.

8. The method of claim 1, wherein the indication of the shifted boundary comprises intraoperatively obtained images of the targeted tissue portion.

9. The method of claim 1, wherein the indication of the shifted boundary includes an indication of an excision boundary between excised and non-excised tissue, and the second working volume is defined using the excision boundary as a fiducial mark.

10-14. (canceled)

15. The method of claim 1, wherein the second working volume is defined by one or more 2-D perimeters corresponding to 2-D cross-sections of the targeted tissue portion.

16-19. (canceled)

20. The method of claim 1, wherein the imaging is performed using an ultrasound imager.

21. The method of claim 1, wherein the excising proceeds through a series of layers, stacked along a longitudinal axis of the surgical tool.

22-23. (canceled)

24. A system for automatic excision of a tissue portion within neural tissue, the system comprising:
a robotically operated surgical tool;
a robotic controller configured to:
receive an excision plan comprising a defined working volume, and
automatically operate the surgical tool to excise tissue within the working volume;
an imager, operable to obtain images of the tissue portion during operation of the surgical tool to excise tissue; and
a target tissue tracker comprising a processor programmed to:
define, using images obtained with the imager, a changed position of an un-excised fraction of the tissue portion after movement of the un-excised fraction due to operation of the robotically operated surgical tool,
generate, based on the changed position, a new excision plan comprising a new working volume including the un-excised fraction of the tissue portion, and
provide the robotic controller with the new excision plan to continue automatic operation of the surgical tool to excise tissue within the new working volume.

25. The system of claim 24, comprising a user interface, wherein the target tissue tracker also defines the changed position of the tissue portion using user-generated selections guided by the images obtained with the imager.

26. The system of claim 24, wherein the robotically operated surgical tool comprises a longitudinally extended device having a flexible and steerable tip with a cutting end.

27. The system of claim 26, wherein the imager is rotatingly coupled to the robotically operated surgical tool to allow rotation of the imager around a longitudinal axis of the surgical tool.

28. (canceled)

29. The system of claim 27, comprising a cannula through which the surgical tool inserts to the tissue portion; wherein the imager is attached to an outer surface of the cannula.

30. The system of claim 27, comprising a cannula through which the surgical tool inserts to the tissue portion; wherein the imager is held within an inner lumen of the cannula.

31. The system of claim 27, comprising a cannula having a laterally positioned window, sized to allow a distal tip of the surgical tool protrude through.

32. The system of claim 24, wherein the imager is an ultrasound imager.

33. The system of claim 24, wherein the imager images the tissue portion through a selectable planar section thereof, and the robotic controller is configured by the excision plan to move the surgical tool through a pattern that excises tissue throughout a first selected planar section imaged by the image, before moving to excise tissue in an at least second selected planar section.

34. (canceled)

35. The system of claim 33, wherein, within each selected planar section, the robotic controller is configured to move the surgical tool distally and proximally along a longitudinal axis of the tissue portion, and radially outward, while successively excising a plurality of layers of tissue.

36-39. (canceled)
